SEQUENCE LISTING (2 sequences)

SEQ ID NO: 1 - DNA, 342 bp, Human cytomegalovirus strain Ad169
tctcttctagaatggctcggaggctatggatcttgagcttactagccgtgaccttgacggtggctttggcggcaccttctcagaaatcgaagcgcagcgtgacggtggaacaacccagtaccagcgctgatggtagtaataccacccccagcaagaacgtaactctcagtcagggggggtccaccaccgacggagacgaagattactccggggagtatgacgttttgattacagacggagatggcagcgaacatcagcaaccacaaaagactgatgaacacaaagaaaatcaagccaaagaaaatgaaaagaagattcagtaacagcagacccggatccaga

SEQ ID NO: 2 - protein, 103 aa, Human cytomegalovirus strain Ad169
MARRLWILSLLAVTLTVALAAPSQKSKRSVTVEQPSTSADGSNTTPSKNVTLSQGGSTTDGDEDYSGEYDVLITDGDGSEHQQPQKTDEHKENQAKENEKKIQ

(19) United States
(12) Patent Application Publication
(10) Pub. No.: US 2004/0241163 A1
(43) Pub. Date: Dec. 2, 2004

(54) Compositions and methods for treating inflammation and related conditions

(76) Inventors: Thomas Shenk, Princeton, NJ (US); Dai Wang, North Plainfield, NJ (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/473,757

(22) PCT Filed: Apr. 5, 2002
(86) PCT No.: PCT/US02/11004

Related U.S. Application Data
(60) Provisional application No. 60/281,582, filed on Apr. 5, 2001.

(51) Int. Cl. A61K 39/395

(52) U.S. Cl. 424/145.1

(57) ABSTRACT

Novel pharmaceutical compositions and treatment methods are disclosed for chemokine-mediated inflammatory and immune-related pathological conditions. The pharmaceutical preparation comprises a chemokine binding activity from a supernatant of human cytomegalovirus (HCMV)-infected cells involving the secreted HCMV UL21.5 protein or functional fragment thereof and capable of binding the chemokine RANTES. Methods of treatment comprise administering a therapeutically effective amount of the UL21.5 activity-containing pharmaceutical preparation.

This application claims benefit of U.S. Provisional Application Ser. No. 60/281,582, filed Apr. 5, 2001, the entirety of which is incorporated by reference herein.

Pursuant to 35 U.S.C. §202(c), it is acknowledged that the United States Government has certain rights in the invention described herein, which was made in part with funds from the National Institutes of Health, Grant No. CA85786.

### FIELD OF THE INVENTION

The present invention related generally to the field of immunology and treatment of dysfunctional immune responses. More specifically, the invention relates to therapeutic compositions and methods for using human cytomegalovirus UL21.5 protein, subdomains of the protein, modified derivatives of the protein or biological complexes that contain UL21.5 protein, its subdomains or derivatives, to treat cytokine-mediated inflammatory and immune-related pathological conditions.

### BACKGROUND OF THE INVENTION

The inflammatory response is an attempt by the body to restore or maintain homeostasis after injury or infection. Inflammation is a cellular immune response involving three major processes (1) increased blood supply to the affected area, (2) dilation and increased permeability of blood vessels, and (3) migration of leukocytes and other inflammatory cells from the blood vessels into the surrounding tissue. Acute inflammation is a localized, protective response to infection or injury. However, excessive or chronic inflammation can lead to tissue destruction.

The movement of inflammatory cells from the bloodstream to affected tissue is mediated by a variety of chemoattractant molecules. The chemoattractant cytokines, or chemokines, are a family of molecules that mediate acute and chronic inflammation by attracting inflammatory cells to a site of injury. Chemokines are small, structurally-related molecules that regulate cell trafficking of various leukocytes through interaction with a subset of seven-transmembrane G protein-coupled receptors (GPCRs) (Zlotnik & Yoshie, Immunity 12: 121-127, 2000).

Because motility is key to their function, chemokines play a central role in leukocyte physiology by controlling basal and inflammatory trafficking (Gerard & Rollins, Nature Immunol. 2: 108-115, 2001). The effect of chemokine activation goes beyond the control of locomotion however; it affects granule exocytosis, gene transcription, mitogenesis and apoptosis (Thelen, Nature Immunol. 72: 129-134, 2000). Additionally, chemokine receptors are expressed on many other cell types, including endothelial cells, smooth muscle cells, stromal cells, neurons and epithelial cells, indicating a role for chemokines in a wide variety of tissues (Gerard & Rollins, 2001, supra).

Chemokine activation is essential for normal cellular immune responses. However, chemokine activation has been associated with a wide variety of pathological conditions, particularly in connection with disorders associated with leukocyte infiltration of tissue. These include (1) autoimmune diseases such as rheumatoid arthritis, systemic lupus, erythematosis and multiple sclerosis, (2) graft or transplant rejection, (3) infection-related disorders such as acute and chronic bacterial and viral infections (notably HIV and mycobacteria) and sepsis, (4) inflammatory or allergic disorders such as asthma, arthritis, colitis and psoriasis, (5) neoplasia, including leukocyte recruitment in cancer and angiogenesis, and (6) vascular disease, including atherosclerosis, hypertension and ischemia-reperfusion (Gerard & Rollins, 2001, supra).

The involvement of chemokines in such a wide variety of inflammatory and immune disorders makes them attractive targets for therapeutic intervention. Another therapeutically attractive feature of chemokines is their specificity. Unlike cytokines, which have a pleiotropic effect, chemokines target specific leukocyte subsets and, in some instances, may only attract cells without activating them (Gerard & Rollins, 2001, supra). Further specificity may also be achieved by focusing therapeutic intervention on a subset of chemokines or a single chemokine that selectively attracts or activates an even smaller subset of cells, by virtue of its interaction with certain chemokine receptors, but not others.

A chemokine that presents a particularly attractive therapeutic target is RANTES (regulated upon activation, normal T cell expressed and secreted). RANTES is involved in the generation of inflammatory infiltrates and inhibition of entry of human immunodeficiency virus (HIV-1) into immune cells (Krensky, ACI International 11: 16-21, 1999). RANTES binds several chemokine receptors, including CCR1, CCR3, CCR5 and CCR10 (Id.). It also binds the human cytomegalovirus (HCMV) US28 protein, which has been characterized as a CCR-type chemokine receptor analog. RANTES is expressed in a diverse group of inflammatory diseases, including transplant rejection, arteriosclerosis, rheumatoid arthritis, delayed type hypersensitivity reactions, asthma, endometriosis and cancer (Pattison et al., Clin. Immunotherapy 4: 1-8, 1995). Because of its delayed expression in T lymphocytes (3-5 days after activation) RANTES is believed to play a key role in maintaining and amplifying an ongoing inflammatory response (Krensky, 1999, supra). Accordingly, downward modulation of RANTES activity may be particularly effective for the treatment of chronic or delayed inflammatory conditions.

Though a variety of anti-inflammatory agents are currently available, there is a substantial unmet need for agents with the potency and specificity required to effectively ameliorate inflammatory dysfunction without unwanted side effects. For instance, few anti-inflammatory drugs are direct inhibitors of cellular infiltration, a primary underlying cause of tissue damage associated with inflammation.

Because of the specificity they promise, agents that act as chemokine antagonists are of interest for development as drugs for inhibiting cellular infiltration and resulting pathological conditions associated with inflammatory and autoimmune diseases. Indeed, in the world of nature, a number viruses utilize the strategy of chemokine or chemokine receptor mimicry as a mechanism to avoid triggering an inflammatory response to viral infection. In particular, many of the poxviruses, herpesviruses (including HCMV) and lentiviruses (including HV) encode gene products that interfere with chemokine-mediated signal transduction by a variety of means, including, among others, binding and sequestering free chemokines, thereby rendering them unable to bind cellular receptors.

Viral chemokine binding proteins have been proposed for use as cytokine and chemokine antagonists. For instance, Smith et al. (U.S. Pat. No. 5,359,039) disclose the use of a Cowpox virus A53R-equivalent protein as an antagonist for Tumor Necrosis Factor (TNF). McFadden et al. (U.S. Pat. No. 5,834,419) disclose a method of using a type I chemokine binding protein from poxvirus (the gene product of myxoma virus T7 gene) as an anti-inflammatory agent. This protein was reported to mimic the interferon-γ receptor and to bind several chemokines, including IL-8, MIP-1β and RANTES. Smith et al. (U.S. Pat. No. 5,871,740) disclose methods of using the poxvirus P35 gene product as a chemokine binding agent. The p35 gene product was reported to bind several chemokines, including MCP-1, MCP-3, RANTES, Eotaxin, MIP-1α, MIP-1β and C10. Smith et al. (U.S. Pat. No. 6,355,252) disclose a soluble Vaccinia virus protein, designated A41L, which was reported to bind chemokines in the CXC group, but not the CC group.

From the foregoing discussion, it can be seen that, although a few viral chemokine antagonists have been proposed as anti-inflammatory agents, a need exists for the identification and development of additional viral proteins that can act as chemokine antagonists, thus providing utility as inhibitors of chemokine-mediated pathological conditions. In particular, agents capable of modulating the activity of a one or a subset of chemokines, as opposed to a broad group of chemokines, are presently unavailable. These would be of particular utility in providing a specific anti-inflammatory effect with minimal side-effects. Targeting of RANTES would be particularly advantageous in view of its suspected role in delayed amplification of the inflammatory response.

### SUMMARY OF THE INVENTION

The present invention provides novel formulations and methods for treating inflammatory diseases and other pathological conditions associated with aberrant chemokine mediation of biological responses in the body.

According to one aspect of the invention a method of binding a RANTES chemokine is provided. The method comprises contacting the RANTES chemokine with a composition comprising a secreted HCMV UL21.5 protein or a functional fragment or subdomain, modified derivative of the protein or biological complex that contains the UL21.5 protein, functional fragment or derivative. In one embodiment, this RANTES-binding UL21.5-associated activity is obtained from supernatants of HCMV-infected cells.

Another aspect of the invention features a pharmaceutical preparation for treating a cytokine-associated inflammatory or immune pathological condition, which comprises the aforementioned UL21.5 activity or one or more genes encoding proteins that provide the activity; e.g. UL21.5 or functional fragments or derivatives thereof. In a preferred embodiment, the pharmaceutical preparation binds a subset of chemokines that includes RANTES. In other embodiments, the pharmaceutical preparation also contains one or more additional anti-inflammatory or immunomodulatory agents.

According to another aspect of the invention, a method of treating a cytokine-associated inflammatory or immune pathological condition in a patient in need of such treatment is provided. The method comprises administering to the patient a therapeutically effective amount of the aforementioned pharmaceutical preparation. The method is used preferably for RANTES-mediated pathological conditions, though it can be used for pathologies associated with any cytokine that is antagonized by the UL21.5 activity described herein. In preferred embodiments, the method is used to treat conditions such as transplant rejection, arteriosclerosis, rheumatoid arthritis, delayed type hypersensitivity reactions, asthma, endometriosis and cancer. In other preferred embodiments, the method is used in combination with other treatment methods, including administration of other anti-inflammatory or immunomodulatory agents.

Other features and advantages of the present invention will be understood by reference to the drawings, detailed description and examples that follow.

### BRIEF DESCRIPTION OF THE DRAWINGS

FIG. 1. Autoradiograms showing results of a chemokine binding assay of HCMV-infected cell supernatants with RANTES, MIP-1 α and IL-8 (FIG. 1A) and a competition assay (FIG. 1B) with RANTES, MIP-1α and MCP-1. Experiments are described in Example 2. Polypeptide molecular weights are indicated at the left side of each set of autoradiograms. Mock=supernatants from mock-infected cell cultures; Wt=supernatants from cell cultures infected with wild-type HMCV; ΔUL21.5=supernatants from cell cultures infected with mutant HCMV deleted for functional UL21.5 gene.

FIG. 2. Autoradiogram showing a competition assay with RANTES, IFN-γ and IFN-α using HCMV-infected cell supernatants. Experiments are described in Example 3. Polypeptide molecular weights are indicated at the left side of the autoradiogram. Concentrations of respective cytokines/chemokines are shown above the autoradiogram. The location of free RANTES is indicated with an arrow.

FIG. 3. Graphs showing a plot of Kd of HCMV-infected cell supernatant for RANTES. Experiments are described in Example 4.

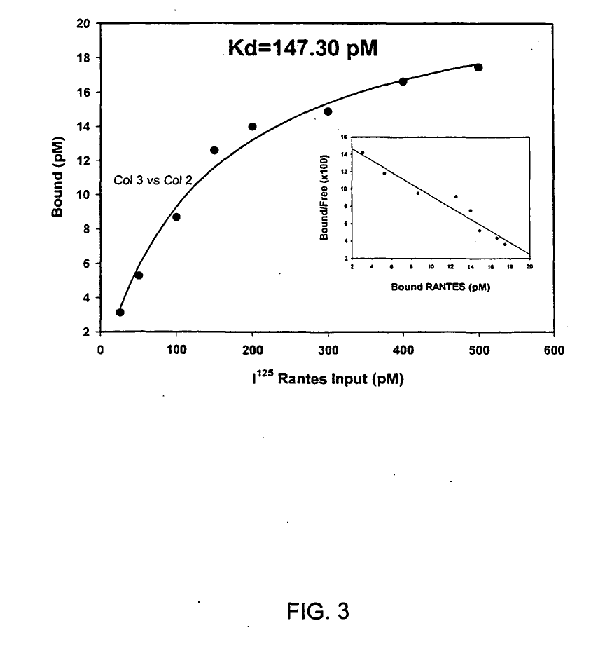

FIG. 4. Graph showing the effect of UL21.5 in HCMV-infected cell supernatant on the ability of RANTES to bind to its cellular receptors. Experiments are described in Example 5. Mock=supernatants from mock-infected cell cultures; Wt=supernatants from cell cultures infected with wild-type HMCV; ΔUL21.5=supernatants from cell cultures infected with mutant HCMV deleted for functional UL21.5 gene.

### DETAILED DESCRIPTION OF THE INVENTION

The inventors have discovered a novel source of anti-inflammatory agents for treating a wide variety of pathological conditions associated with cytokine-mediated trafficking of leukocytes and other immunoresponsive cells during inflammation and immune responses to injury, infection or disease. These agents are found in the supernatant of cells infected with human cytomegalovirus, which comprises the secreted glycoprotein encoded by the cytomegalovirus UL21.5 gene.

Human cytomegalovirus (HCMV) is a herpesvirus belonging to the Betaherpesviritnae subgroup. HCMV infection is normally proinflammatory; however, HCMV is known to produce multiple immune evasion polypeptides, including two CXC chemokine agonists called vCXC-1 and vCXC-2 (Renfold, Proc. Natl. Acad. Sci. USA 96: 9839-9844, 1999) and a GPCR, encoded by the US28 gene, which is specific for several CC chemokines and the CX3C chemokine fractalkine (Gao & Murphy, J. Biol. Chem. 269: 28539-28542, 1994; Kledal et al., FEBS Lett. 441: 209-214, 1998).

In accordance with the present invention, it has now been discovered that the HCMV UL21.5 gene product is also involved in chemokine binding. The HCMV UL21.5 gene encodes a glycoprotein of heretofore unknown function, which is abundantly secreted from HCMV-infected cells (Müllberg et al., J. Gen. Virol. 80: 437-440, 1999). The deduced product of the UL21.5 gene comprises a highly glycosylated protein 103 amino acids in length, with no apparent homology to any other viral or cellular protein. Two secreted forms of the protein are found, of 45 kDa and 25 kDa, respectively (Müllberg et al., 1999, supra). The UL21.5 protein sequence is highly conserved among clinical isolates of HCMV (see Scott et al., 2001, GenBank Accession Nos. AF413626 through AF413645, inclusive) and the presence of UL21.5 mRNA is significantly associated with the occurrence of HCMV disease (Andre et al., Diagn. Microbiol. Infect. Dis. 34: 287-291, 1999), both factors indicating that the protein plays a key role in HCMV infection.

Western blot analysis of supernatants of HCMV-infected human fibroblasts shows that UL21.5 accumulates between about 72 and 96 hours post-infection. By two independent means, described in Examples 2 and 3, the inventors have shown that these infected cell supernatants bind the chemokine RANTES, by a mechanism that involves the UL21.5 protein or domains of the protein, modified derivatives of the protein or biological complexes that contain UL21.5 or domains or derivatives thereof. Supernatants of cells infected with a HCMV mutant comprising a UL21.5-deleted genome do not bind RANTES. Significantly, UL21.5-containing cell supernatant was found to be specific for RANTES, inasmuch as it was found not to bind other cytokines or chemokines tested by the inventors, including MIP-1α , MCP-1 (both CC chemokines) IL-8 (a CXC chemokine), IFN-α and IFN-γ. Further, the binding affinity of UL21.5-containing supernatants for RANTES is high: Kd=150 pM, which is comparable to or up to tenfold higher than the binding affinity of RANTES for its cellular receptor(s) (Gong et al., J. Biol. Chem. 271: 10521-10527, 1996).

In one aspect, the present invention provides novel compositions and methods for binding selected chemokines. In particular, the compositions are selective for RANTES. The compositions may also be selective for other chemokines; however, the inventors have determined that the compositions do not bind several other cytokines and chemokines, as mentioned above.

It can be seen from the foregoing summary that one or more products of the HCMV UL21.5 gene in supernatants of HCMV-infected cells is required for the chemokine binding activity observed in accordance with the present invention. One or more additional components of HCMV-infected cell supernatants may also be required or desired for optimum chemokine binding. Accordingly, a preferred embodiment of the present invention is drawn to a chemokine-binding composition that comprises UL21.5 or functional fragments thereof (defined below and used interchangeably with “subdomains”), as well as modified derivatives of the protein or biological complexes that contain UL21.5 or subdomains or derivatives thereof, present within the culture medium of HCMV-infected cells. These substances are sometimes referred to individually or collectively herein as “UL21.5 activity.” Additionally, because the initial preparation of UL21.5 activity comprises centrifugation to remove cells, virus particles and debris from the culture medium, this medium is referred to herein as “HCMV-infected cell supernatant” or UL21.5 activity-containing supernatant.” Thus, term “UL21.5 activity” is intended to refer to the chemokine binding activity exhibited by HCMV-infected cell supernatants, which contain UL21.5 protein or subdomains, modified derivatives or biological complexes containing UL21.5 or subdomains or derivatives thereof. This chemokine binding activity includes high affinity binding of RANTES, and may include binding to a larger subset of chemokines, but does not include high affinity binding to the chemokines MP-1α, MCP-1 and IL-8, or the cytokines IFN-α and IFN-γ

Conditioned medium from HCMV-infected cultured cells is prepared according to routine procedures well known in the art. Since the UL21.5 gene is present in all HCMV, any strain of HCMV is suitable for use in the present invention. Further, as functional homologs of the HCMV UL21.5 are identified in CMVs from other mammals (e.g., mouse, rat, primate), these CMVs and their UL21.5 homologs also will be suitable for use in the present invention.

Any cultured cell type capable of infection by the selected HCMV is suitable for use in the present invention. For instance, a preferred embodiment comprises human fibroblast cells infected with HCMV. Other suitable cell types include, but are not limited to, endothelial cells, muscle cells and lymphocytes.

Methods of infecting cultured cells with virus and collecting the medium in which infected cells are cultured are also well known in the art. An exemplary method, using HCMV infected human fibroblasts, involves the following steps. Human fibroblasts are infected with HCMV at multiplicity of 1-3 pfu/cell using routine procedures. Following the initial infection period (e.g., 1 hour) cells are washed with culture medium (e.g., serum-free Dulbecco's modified Eagle's Medium (DMEM)), followed by addition of fresh medium containing 10% fetal calf serum (FCS). Infection is allowed to proceed for a period of type appropriate to the cell type, e.g., 6-8 hours for human fibroblasts, after which the cells are washed with serum-free medium and placed into fresh serum-free medium. Forty-eight hours after infection, the culture medium is collected and centrifuged at 55,000 g for one hour to remove residual virus. Optionally, the supernatant may be concentrated and the medium exchanged for buffer, e.g., PBS, using standard methods. As another option, the supernatant may be dried or lyophilized . This basic process may be modified according to methods familiar to virologists to accommodate the infection, culture and collection of medium from other cell types.

In an alternative embodiment, the components of HCMV-infected cell supernatants required for cytokine binding may be purified and combined to produce a “reconstituted” cytokine-binding composition. For this and similar embodiments, purified UL21.5 protein or a functional fragment is needed. As used herein, the term “functional fragment” refers to any portion of the UL21.5 polypeptide that possesses the relevant biological activity of the entire polypeptide or subdomains, modified derivatives or biological complexes containing UL21.5, as defined above.

The UL21.5 protein may be purified from supernatants of HCMV-infected cells according to a variety of standard methods, including for example, ammonium sulfate precipitation gel filtration, ion exchange and affinity separation (e.g., immuno-affinity purification).

In preferred embodiments, UL21.5 protein is produced by expression of an isolated UL21.5 gene. The UL21.5 gene from any HCMV is suitable for this purpose, as are gene variants and fragments encoding a functional UL21.5 protein or fragment thereof. An example of a UL21.5 gene and its encoded protein from HCMV strain Ad169 is set forth below as SEQ ID NO:1 and SEQ ID NO:2. Other suitable UL21.5 variants are published as GenBank Accession Nos. AF413627, AF413628, AF413629, AF413630, AF413631, AF413632, AF413633, AF413634, AF413635, AF413636, AF413637, AF413638, AF413639, AF413640, AF413641, AF413642, AF413643, AF413644 and AF413645.

SEQ ID NO: 1 (coding portion is underlined):
tctcttctagaatggctcggaggctatggatcttgagcttactagccgtgaccttgacggtggctttggcggcaccttctcaga
aatcgaagcgcagcgtgacggtggaacaaccagtaccagcgctgatggtagtaataccacccccagcaagaacgtaac
tctcagtcagggggggtccaccaccgacggagacgaagattactccggggagtatgacgttttgattacagacggag
atggcagcgaacatcagcaaccacaaaagactgatgaacacaaagaaaatcagccaaagaaatgaaagaagattc
agtaacagcagacccggatccaga
SEQ ID NO: 2:
MARRLWILSLLAVTLTVALAAPSQKSKRSVTVEQPSTSADGSNTTPSKNVTLS
QGGSTTDGDEDYSGEYDVLITDGDGSEHQQPQKTDEHKENQNKENEKKIQ

According to standard methods, a UL21.5 gene or gene fragment, as well as genes encoding other possible components of a complex exhibiting UL21.5 activity, may be produced by expression in a suitable expression system. For example, part or all of a DNA molecule may be inserted into a mammalian viral vector for expression in mammalian cells, or into a baculovirus vector for expression in an insect cell. Such vectors comprise the regulatory elements necessary for expression of the DNA in the host cell, positioned in such a manner as to permit expression of the DNA in the host cell. Such regulatory elements required for expression include promoter sequences, translation control sequences and, optionally, enhancer sequences. Expression of the gene in a eucaryotic system ensures proper post-transcriptional/translational processing of the mRNA and its encoded protein, including glycosylation of the protein.

The UL21.5 protein produced by gene expression in a recombinant system may be purified according to methods known in the art. In a preferred embodiment, the recombinant protein contains several (e.g., 6-8) histidine residues on the amino or carboxyl termini, which allows the protein to be affinity purified on a nickel column. If histidine tag-vectors are not used, an alternative approach involves purifying the recombinant protein by affinity separation, such as by immunological interaction with antibodies that bind specifically to the recombinant protein. Such methods are commonly used by skilled practitioners.

The UL21.5 compositions of the present invention are generally administered to a patient as a pharmaceutical preparation. The term “patient” as used herein refers to human or animal subjects (animals being particularly useful as models for clinical efficacy of a particular pharmaceutical preparation). Selection of a suitable pharmaceutical preparation depends upon the method of administration chosen, and may be made according to protocols well known to medicinal chemists.

The pharmaceutical preparations of the invention are formulated for administration with a acceptable medium such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of a particular composition in the chosen medium will depend on the hydrophobic or hydrophilic nature of the medium, in combination with the specific properties of the delivery vehicle and active agents disposed therein. Solubility limits may be easily determined by one skilled in the art.

As used herein, “biologically acceptable medium” includes any and all solvents, dispersion media and the like which may be appropriate for the desired route of administration of the pharmaceutical preparation, as exemplified in the preceding paragraph. The use of such media for pharmaceutically active substances is known in the art. Except insofar as any conventional media or agent is incompatible with the compositions to be administered, its use in the pharmaceutical preparation is contemplated.

The pharmaceutical preparation is formulated in dosage unit form for ease of administration and uniformity of dosage. “Dosage unit form,” as used herein, refers to a physically discrete unit of the pharmaceutical preparation appropriate for the patient undergoing treatment. Each dosage should contain a quantity of the UL21.5-containing HCMV-infected cell supernatant calculated to produce the desired protective effect in association with the selected pharmaceutical carrier. Procedures for determining the appropriate dosage unit are well known to those skilled in the art.

Dosage units may be proportionately increased or decreased based on the weight of the patient. Appropriate concentrations for achieving the desired therapeutic effect may be determined by dosage concentration curve calculations, as known in the art.

As one example, for topical applications, e.g., for treating arthritis or an inflammatory skin disorder, the pharmaceutical preparation may be used at an effective concentration of UL21.5 activity in an appropriate carrier (e.g., cream, wax, liposome emulsion) applied to the dermal site. As another example, for gastrointestinal administration, e.g., for treatment of chronic GI inflammatory conditions, the oral dose of the UL21.5 activity-containing medium in an appropriate carrier (e.g., lipid emulsion) is normalized to the lumenal surface area of the stomach and duodenum. As a further example, for inflammation of airways, the pharmaceutical preparation is formulated for aerosol distribution at an effective concentration of UL21.5 activity. As yet another example, e.g., for treatment of rheumatoid arthritis or inflammatory disorders of muscle or other tissue, the pharmaceutical preparation may be prepared as an injectable dose of UL21.5 activity.

It will also be appreciated by persons of skill in the art that pharmaceutical formulations of the invention may contain UL21.5 activity together with one or more anti-inflammatory or immunomodulatory agents. Various combinations of such agents may be useful for certain applications, and formulations of such combinations would be prepared according to the general guidelines set forth above. Moreover, one or more anti-inflammatory or immune-modulatory agents may be combined with other agents, such as analgesics, for example, to provide a pharmaceutical formulation that is effective by two different modes of action.

The pharmaceutical preparations described above are expected to be useful for treating a wide variety of clinical conditions involving disorders in chemokine-medicated trafficking of leukocytes or other inflammatory cells. Such conditions include, but are not limited to, (1) autoimmune diseases such as rheumatoid arthritis, systemic lupus, erythematosis and multiple sclerosis, (2) graft or transplant rejection, (3) infection-related disorders such as acute and chronic bacterial and viral infections (notably IV and mycobacteria) and sepsis, (4) inflammatory or allergic disorders such as asthma, arthritis, colitis and psoriasis, (5) neoplasia, including leukocyte recruitment in cancer and angiogenesis, and (6) vascular disease, including atherosclerosis, hypertension and ischemia-reperfusion.

Due to the specificity of UL21.5 for a subset of chemokines, including RANTES, the pharmaceutical preparations of the invention promise to be of particular utility in the treatment of delayed or chronic inflammatory disorders, and other disorders associated with RANTES expression. These include, but are not limited to, transplant rejection, arteriosclerosis, rheumatoid arthritis, delayed type hypersensitivity reactions, asthma, endometriosis and cancer.

The pharmaceutical preparations of the invention may be administered locally or systemically, depending on the nature of the disorder to be treated. Any suitable route of administration and formulation adapted thereto is considered within the scope of the present invention, including oral, inhalatory, intranansal, topical, transdermal, intraocular, urovaginal, rectal, intraperitoneal, intramuscular, and intravenous administration. The pharmaceutical preparation is administered in a “therapeutically effective” amount, i.e., at a dosage level and frequency and for a time sufficient to alleviate the disorder being treated. As used herein, the term “alleviate” means to reduce or eliminate the symptoms or the underlying cause of the pathological condition. For example, if the symptom is inflammation of tissue, with the underlying cause being infiltration of inflammatory cells such as leukocytes, the condition is alleviated upon reduction in such cellular infiltration and consequent decrease in inflammation of the tissue.

In one embodiment of the invention, the pharmaceutical preparation is administered locally to a site of inflammation. For instance, inflammatory or autoimmune disorders of the skin (e.g., acute or chronic dermatitis, eczema, psoriasis) may be treated by topical administration of the pharmaceutical preparation formulated as a cream, lotion or ointment, at intervals for a period of time sufficient to alleviate the condition. As another example, inflammations of the joints or tendons (e.g., arthritis, tendonitis) may be treated by injecting the pharmaceutical preparation into the affected location. Such injections may be administered at intervals until inflammation has subsided. As yet another example, inflammatory conditions of the airways or lungs (e.g., asthma, emphysema) may be treated by inhalation therapy with an aerosol formulation of the pharmaceutical preparation, which may be used for immediate relief of acute symptoms, or which may be administered regularly over a predetermined time course to treat the disorder. As still another example, inflammations or autoimmune diseases of the gastrointestinal tract (e.g., irritable bowel syndrome, Crohn's Disease) may be treated orally with the pharmaceutical preparation formulated as a liquid to coat the lumenal surface of the gastrointestinal tract. Such a formulation could be administered for acute or chronic symptoms in accordance with standard medical procedures.

In another embodiment, the pharmaceutical preparation is administered systemically for treatment of inflammatory or immune disorders that are, at least in part, systemic in nature (e.g., systemic lupus, multiple sclerosis, rheumatoid arthritis, dermatomyositis and scleroderma), or that do not lend themselves well to localized drug delivery, or as a supplement to localized drug delivery. For example, the formulation may be administered intravenously as a generalized anti-inflammatory or immunomodulatory agent following organ or tissue transplant, vascular surgery, balloon angioplasty or large scale trauma, such as burns. As another example, the pharmaceutical preparation can be administered orally to treat acute or chronic infection, e.g., bacterial or viral infections.

As mentioned hereinabove the UL21.5 protein , or subdomains of the protein, modified derivatives of the protein or biological complexes that contain UL21.5 or its subdomains or derivatives, is a key component of the HCMV-infected cell supernatant, required for binding RANTES. Accordingly, the UL21.5 gene may find therapeutic utility in gene therapy of one or more inflammatory or immune disorders. The gene or genes can be administered as naked DNA or as DNA in complexes that enhance its delivery for gene therapy purposes. Numerous vectors are available for use in gene therapy protocols to deliver the gene(s) needed to produce UL21.5 activity. These include, but are not limited to, vectors based on parvoviruses, such as adeno-associated virus, adenoviruses, or retroviruses including lentiviruses. The gene or genes that produce UL21.5 activity can be expressed constitutively, or their expression can be regulated by promoters that are active under specific in vivo conditions or that become active in response to specific inducers that can be administered to the patient.

As mentioned, combination therapy may be appropriate in any of the foregoing methods of treatment. That is, the pharmaceutical preparations of the invention may be combined with other anti-inflammatory or immunomodulatory drugs or therapies to achieve the desired result.

The following examples are set forth to describe the invention in greater detail. They are intended to illustrate, not to limit, the invention.

### EXAMPLE 1

### Preparation of Experimental Materials

Construction of UL21.5 Knock Out Virus.

The green fluorescent protein (GFP)/internal ribosomal entry site (IRES)/puromycin (Puro) cassette controlled by the simian virus 40 promoter and poly(A) site was substituted for the UL21.5 open reading frame. The knockout virus is referred to as ADsubUL21.5 (ΔUL21.5).

Preparation of UL21.5-Containing Supernatants from HCMV-Infected Human Fibroblasts.

Human fibroblasts cells were infected at multiplicity of 1-3 pfu per cell, with Adwildtype (wt) or ADsubUL21.5 (ΔUL21.5) virus, or with vehicle (mock infection). After 1 hour, the cells were washed once with serum-free Dulbecco's modified Eagle's medium (DMEM), and then fresh DMEM containing 10% fetal calf serum (FCS) was added. Infection was allowed to proceed for 6-8 h, and the cells were then washed twice with serum-free DMEM, and fresh medium without serum was added. Forty-eight hours after infection, the culture medium was collected, and was then centrifuged at 55,000 g for 1 h to remove residual free virus. Subsequently, the supernatants were concentrated 300 fold and buffer exchanged to phosphate-buffered saline with Centriprep-10.

### EXAMPLE 2

### HCMV Encodes a Secreted Chemokine Binding Protein

Chemokine Binding Assay.

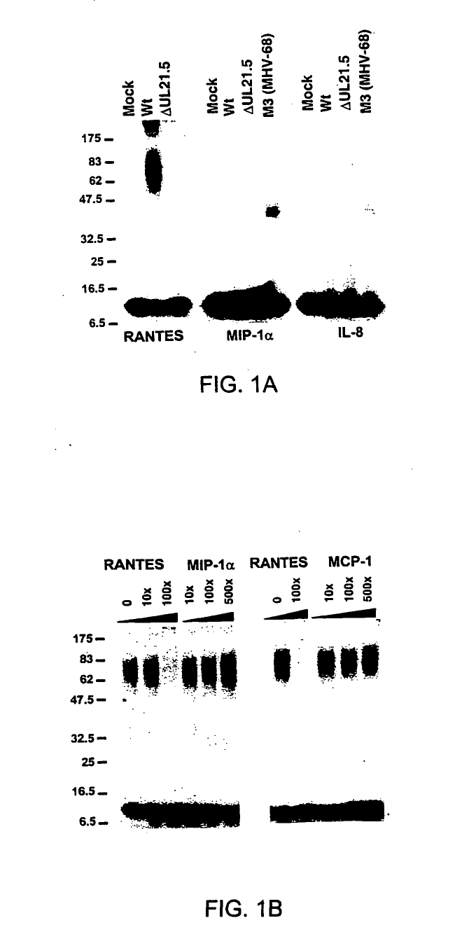

10 μl of concentrated medium from wild type and ADsubUL21.5 virus infected human fibroblasts (equivalent to 2×10⁴ cells) was incubated with ¹²⁵I-labeled chemokines (0.4 nM of RANTES, MIP-1α or IL-8) for 2 hours at room temperature. In several cases purified murine herpesvirus-68 (MHV-68) M3 chemokine-binding protein was included as a positive control. The chemokine-protein complexes that formed were then covalently cross-linked by the addition of 1-ethyl-3-(3-dimethyl-aminopropyl) carbodiimide (EDC) to a final concentration of 40 mM for 30 min at room temperature. The reaction was quenched by the addition of 1/10 volume of 1M Tris-HCl (pH7.4). The crosslinked chemokine-protein complex was analyzed by 10% SDS-PAGE, as shown in FIG. 1A. The major UL21.5-RANTES complex migrates at 62-83 kDa, the M3-MIP-1α and M3-IL-8 complexes migrate at about 40 kDa, and free RANTES migrates near the bottom of the gels. The results shown in FIG. 1A indicate that UL21.5 binds to RANTES, but not at a detectable level to MIP-1α or IL-8.

Binding Specificity of UL21.5.

Competition assays were performed using concentrated medium from infected human fibroblasts as a source of UL21.5 activity, in a manner similar to the cross-linking experiment described above, except that 10-500 fold excesses of unlabeled chemokines (RANTES, MIP-1α or MCP-1) were included in the assays as competitors for binding of ¹²⁵I-labeled RANTES to UL21.5 protein. Results are shown in FIG. 1B. These results corroborate the results of the cross-linking experiment described above, showing that UL21.5 binds to RANTES, but not to MIP-1α, and further demonstrating that UL21.5 also does not bind at a detectable level to MCP-1.

### EXAMPLE 3

### UL21.5 Does Not Bind to Interferons

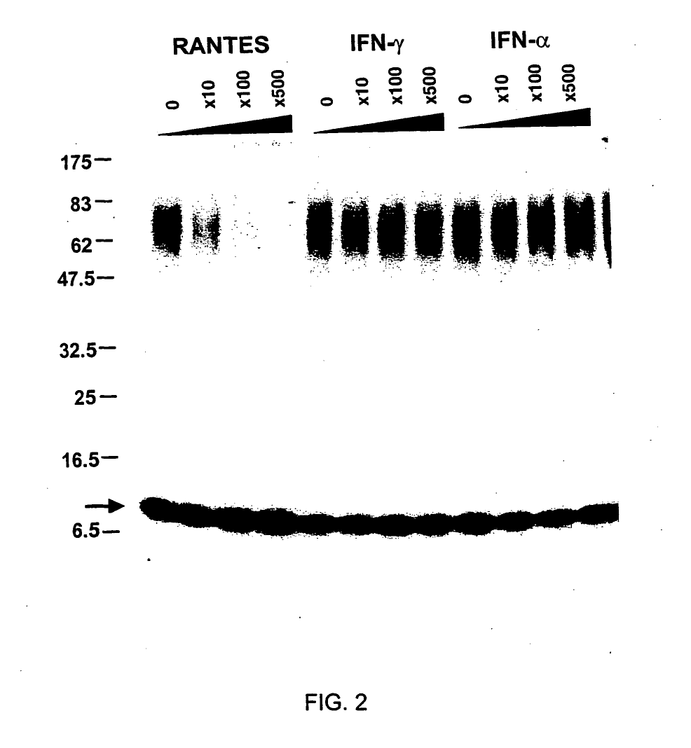

A competition experiment was performed as described in Example 2, using ¹²⁵I labeled RANTES with unlabeled RANTES, interferon-γ (IFN-γ) or interferon-α (IFN-α) as competitors. Results are shown in FIG. 2. This experiment confirms the results of the experiments performed in Example 1, demonstrating that UL21.5 binds to RANTES. The results also show that UL21.5 does not bind at a detectable level to interferon-γ or interferon-α.

### EXAMPLE 4

### UL21.5 Protein Binds to RANTES with High Affinity

A Scatchard analysis of ¹²⁵I-labeled RANTES binding to UL21.5 protein was performed. Aliquots of concentrated medium from cultures of wild type and ADsubUL21.5 virus infected human fibroblasts (2×10⁴ cell equivalents) were incubated with different concentrations (25-500 pM) of ¹²⁵I-RANTES for 2 h at room temperature. The complex was precipitated with 20% polyethylene glycol and filtered through Whatman GF/C filters (method described by Alcami & Smith, Cell 71 153-167, 1992; Alcami, et al., J. Immunol. 160: 624-33, 1998). The filters were washed twice and the radioactivity was determined by using a scintillation counter. Background radioactivity precipitated in the presence of binding medium was subtracted. The apparent K_{d} for the RANTES-UL21.5 interaction was 147.3 pM. This experiment demonstrates that the UL21.5 activity present in the medium of infected cells binds with high affinity to RANTES.

### EXAMPLE 5

### UL21.5 Blocks RANTES Binding to its Cellular Receptor(s)

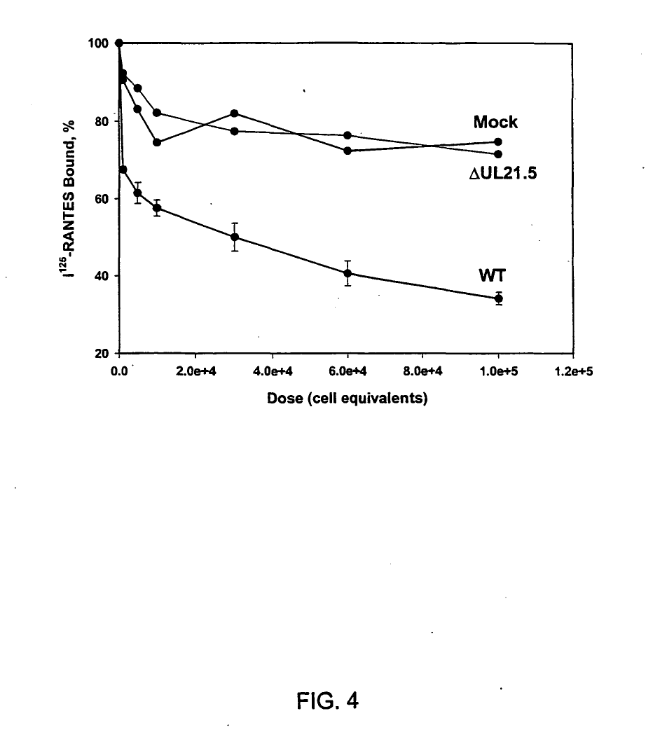

Culture media from wt infected, ADsubUL21.5 infected or mock infected human fibroblasts collected as described in Example 1 were preincubated with 100 pM ¹²⁵I-labeled RANTES in 100 μl for 1 hour at 4° C. Subsequently, 2×10⁶ THP-1 cells, which contain the receptor for RANTES were added in 50 μl, and incubated for 2 h at 4° C. After incubation, the cells were centrifuged through 15% sucrose RPMI-1640 culture medium with 0.1% bovine serum albumen, and washed twice. Bound ¹²⁵I-labeled RANTES was quantified by using a scintillation counter. Results are shown in FIG. 4. This experiment demonstrates that the UL21.5 activity present in the medium of HCMV infected cells can block the ability of RANTES to bind to the surface of cells that contain its receptor.

The present invention is not limited to the embodiments described and exemplified above, but is capable of variation and modification without departure from the scope of the appended claims.

We claim:

1. A pharmaceutical preparation for treating a chemokine-associated inflammatory or immune pathological condition, the preparation comprising a secreted HCMV UL21.5 protein or a functional fragment thereof that binds a RANTES chemokine, in a biologically acceptable medium.

2. The pharmaceutical preparation of claim 1, wherein the chemokine-associated inflammatory or immune pathological condition is a RANTES-associated condition.

3. The pharmaceutical preparation of claim 1, comprising a concentrated supernatant of HCMV-infected cells.

4. The pharmaceutical preparation of claim 3, comprising a dried supernatant.

5. The pharmaceutical preparation of claim 3, wherein the HCMV-infected cells are selected from the group consisting of endothelial cells, muscle cells and lymphocytes.

6. The pharmaceutical preparation of claim 3, wherein the HCMV-infected cells are fibroblast cells.

7. The pharmaceutical preparation of claim 1, comprising one or more genes encoding one or more polypeptides having UL21.5 activity.

8. The pharmaceutical preparation of claim 1, further comprising one or more additional anti-inflammatory agents.

9. The pharmaceutical preparation of claim 1, further comprising one or more additional immune-modulatory agents.

10. The pharmaceutical preparation of claim 1, formulated for treatment of a pathological condition selected from the group consisting of transplant rejection, arteriosclerosis, rheumatoid arthritis, delayed type hypersensitivity reactions, asthma, endometriosis and cancer.

11. The pharmaceutical preparation of claim 1, formulated for administration by a route selected from the group consisting of oral, intranansal, topical, urovaginal, rectal, intraperitoneal, intramuscular, and intravenous.

12. A method of binding a RANTES chemokine, comprising contacting the RANTES chemokine with a supernatant from cultured cells infected with HCMV, wherein the supernatant comprises a secreted HCMV UL21.5 protein or a functional fragment thereof.

13. The method of claim 12, wherein the cells are selected from the group consisting of endothelial cells, muscle cells and lymphocytes.

14. The method of claim 12, wherein the cells are fibroblast cells.

15. A method of treating a cytokine-associated inflammatory or immune pathological condition in a patient in need of the treatment, the method comprising administering to the patient a therapeutically effective amount of a pharmaceutical preparation comprising a HCMV UL21.5 protein or a functional fragment thereof that binds a RANTES chemokine, in a biologically acceptable medium.

16. The method of claim 15, wherein the patient is a human.

17. The method of claim 15, wherein the patient is a non-human mammal.

18. The method of claim 15, wherein the cytokine-associated inflammatory or immune pathological condition is a RANTES-associated condition.

19. The method of claim 15, wherein the pathological condition selected from the group consisting of transplant rejection, arteriosclerosis, rheumatoid arthritis, delayed type hypersensitivity reactions, asthma, endometriosis and cancer.

20. The method of claim 15, wherein the pharmaceutical formulation is administered by a route selected from the group consisting of oral, inhalatory, intranansal, topical, transdermal, intraocular, urovaginal, rectal, intraperitoneal, intramuscular, and intravenous.

21. The method of claim 15, wherein the treating further comprises administering one or more additional anti-inflammatory agents.

22. The method of claim 15, wherein the treating further comprises administering one or more additional immune-modulatory agents.

23. The method of claim 15, wherein the treatment is provided by administering a therapeutically effective amount of a pharmaceutical preparation comprising one or more genes encoding one or more proteins which, alone or combined, comprise a HCMV UL21.5 activity, the activity being characterized by binding the chemokine RANTES.

24. The method of claim 23, wherein the one or more genes are inserted into a vector customized for expressing the one or more genes in a mammalian cell.
